(19) United States
(12) Patent Application Publication
(10) Pub. No.: US 2007/0249906 A1
(43) Pub. Date: Oct. 25, 2007

(54) Self-propelled endoscopic device with anchor system

(75) Inventors: Samuele Gorini, Pisa (IT); Alberto Arena, Cascina (IT); Giuseppe Pernorio, Pisa (IT); Arianna Menciassi, Pisa (IT); Paolo Dario, Livorno (IT)

Correspondence Address: POLLACK, P.C., THE CHRYSLER BUILDING, 132 EAST 43RD STREET, SUITE 760, NEW YORK, NY, 10017, US

(73) Assignee: Era Endoscopy S.r.l.

(21) Appl. No.: 11/648,011

(22) Filed: Dec. 28, 2006

(30) Foreign Application Priority Data
Dec. 28, 2005 (EP) 05425927.0

(51) Int. Cl. A61B 1/005

(52) U.S. Cl. 600/139; 600/101

(57) ABSTRACT

A self-propelled endoscopic device with anchor system, comprising a tubular body of variable length extending between front end and rear end sections with respective anchors for securing the ends, temporarily and alternately, to a wall of a body cavity in synch with corresponding axial contractions and elongations of the tubular body that result from injection of compressed air and, hence, creation of a vacuum therein. Also provided is a pneumatic actuator of a mobile jaw of the anchors sliding axially on a multichannel support, wherein at least one suction hole is formed for generating a vacuum suitable for drawing surrounding tissue of the body cavity into the anchors. A relatively evenly perforated diaphragm is placed, coaxially with the support, at the suction hole and in a spaced relationship from the surface of the support, the width of the perforations being generally smaller than that of the suction hole.

### FIELD OF THE INVENTION

The present invention relates generally to medical devices and, more particularly, to a device suitable for locomotion through a body cavity.

### BACKGROUND OF THE INVENTION

Endoscopic devices are typically used by surgeons for a variety of surgical and/or diagnostic procedures. In operation, the surgeon applies manual force, for instance, directly to the device in order to impart forward motion of the device through a patient's body. Such devices are usually operated in conjunction with other surgical and/or diagnostic instruments, e.g., micro-arms, micro-cameras and/or laser emitters, that may be needed to complete various medical procedures.

Endoscopic devices of this type, but capable of autonomous or semi-autonomous locomotion through the body cavity of a patient, are described, for instance, in U.S. Pat. No. 5,398,670, U.S. Pat. No. 5,906,591 and WO02/068035. The endoscopic device described in these documents substantially consists of a tubular body of variable length with two end portions, said front and rear end portions, comprising anchoring means that enable the front end portion and the rear end portion to become temporarily and alternately attached to the wall of the body cavity, for example the intestine, to enable the forward motion of the device.

In particular, the variable-length tubular body of the endoscopic device described in the above-mentioned documents is in the form of a bellows-shaped tubular body and is consequently capable of extending and contracting as a result of admission and respectively aspiration of air therein. More specifically, during the extension phase, the bellows are pressurized by means of compressed air, obtaining an elongation proportional to the pressure injected, while the bellows are contracted by progressively reducing the pressure inside therein, until some degree of vacuum is created.

In the above-mentioned patent application PCT no. WO02/068035, the device is anchored to the wall of the body cavity by clamp means associated with the front and rear end portions of the device and selectively enabled by an external control unit in synchronism with the successive extensions and contractions of the bellows-shaped tubular body. The aforementioned clamp means are enabled by pneumatic actuating means which, in the preferred embodiment described in the aforesaid document, also consist of bellows-shaped members.

More particularly the clamp means comprise a pneumatically actuated mobile member and a fixed member. The mobile member, moving towards the fixed one, traps the tissue bordering the body cavity, achieving the grip necessary for locomotion. In order to allow the tissue to be drawn between the two members, a localised vacuum is generated between the latter which causes the surrounding tissue to collapse between them. This vacuum is generated by aspiration through a hole formed between the two gripping members and communicating with an external aspiration system.

A first problem found in the anchorage system provided in the endoscopic device according to patent application no. WO/02/068035 lies in the fact that, in order to create localised vacuum, debris may be also sucked together with air, which, if larger in size than the suction hole, may obstruct the same hole, thereby preventing the surrounding tissue from collapsing between the two gripping members and making the anchoring action of the device to the wall of the body cavity partially or wholly ineffective.

A further problem relates to the fact that, when the tissue, collapsing between the two gripping members reaches the suction hole, the latter is blocked, so that only a part of the surrounding tissue is effectively sucked in between the two gripping members. Consequently the tissue is partially gripped, which may be ineffective for the locomotion phase in that, during the phase of elongation or contraction of the tubular body of the endoscopic device, disengagement of the tissue may occur, with consequent loss of grip by the front end or the rear end of the device, thus creating an inefficient locomotion movement.

Moreover, when the tissue reaches the suction hole, obstruction of the hole is accompanied by the entry of a portion of tissue in said hole. Due to the long suction action lasting throughout the phase of elongation or contraction of the tubular body, and to its high intensity (−0.7 bar), the edge of the hole may damage the mucosa and to some underlying tissue layers with consequent flare and, at times, bleeding.

### OBJECTS AND SUMMARY OF THE INVENTION

Accordingly, it is an object of the present invention to provide a self-propelled endoscopic device having a system for temporary and alternating anchoring of front and rear ends of the device to a wall portion of a patient's body cavity, that is stable and evenly distributed over the entire perimeter of the ends, so as to avoid obstruction of a suction hole of the device.

Another object of the present invention is to provide an endoscopic device of the aforementioned type wherein anchorage of its ends to the walls of the body cavity takes place without risks of damage to the tissue involved in the action of anchorage.

These objects are achieved with the endoscopic device according to the present invention whose main features are set forth in claim 1.

The main feature of the endoscopic device according to the invention consists in that, on the multichannel support whereon the mobile member of the anchoring means slides and wherein the suction hole is formed, an evenly perforated coaxial diaphragm is placed in a spaced relationship from the surface of said multichannel support, the width of the perforations being smaller than that of the suction hole. In a preferred embodiment the diaphragm is formed by a hollow cylindrical member on whose lateral surface a plurality of longitudinal apertures are formed, with width smaller than that of said hole and evenly distributed on said surface. The cylindrical member therefore performs the triple function of filter, distributor of the suction effect and spacer separating the tissue from the suction hole.

### BRIEF DESCRIPTION OF THE DRAWINGS

A specific, illustrative self-propelled endoscopic device, according to the present invention, is described below with reference to the accompanying drawings, in which:

FIG. 1 shows schematically a self-propelled endoscopic device with anchor system, according to one aspect of the present invention;

FIG. 2 is an enlarged view taken longitudinally of a front end portion of the device shown in FIG. 1;

FIG. 3 is a sectional view of the device taken along line III-III of FIG. 2;

FIG. 4 is an enlarged sectional view taken longitudinally of a front end portion of the device taken along line IV-IV of FIG. 3; and

FIG. 5 is a perspective view of a filter body for a self-propelled endoscopic device with anchor system, according to another aspect of the present invention.

### DETAILED DESCRIPTION OF THE PREFERRED EMBODIMENTS

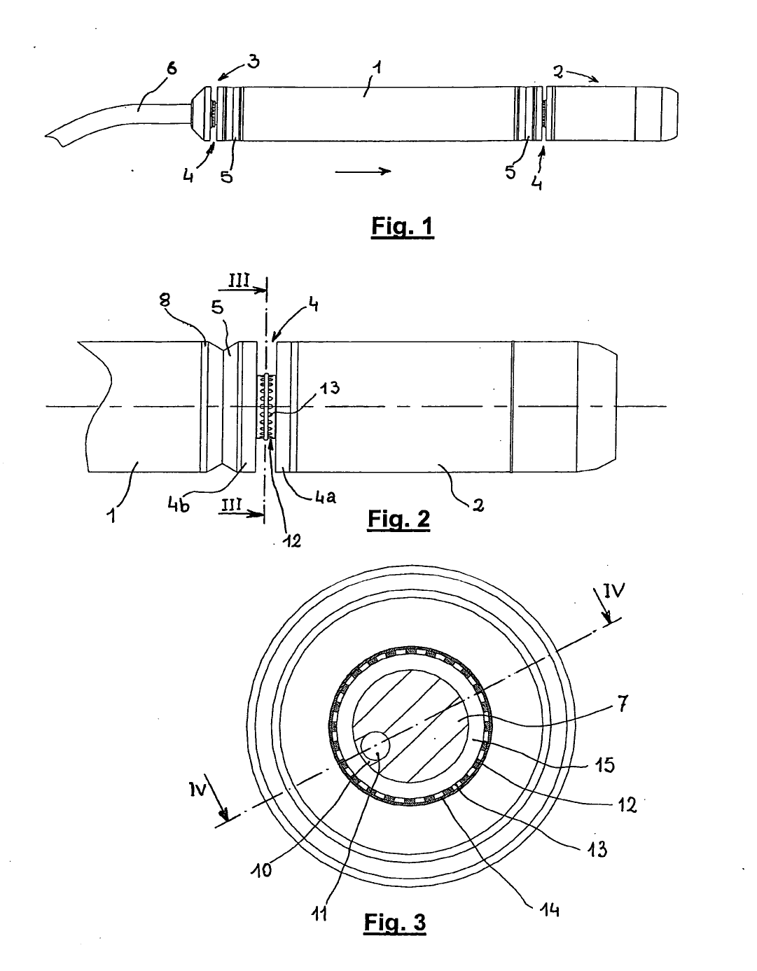

Referring now to the drawings and, more particularly, to FIGS. 1-5, there is shown generally a self-propelled endoscopic device with anchor system, in accordance with various aspects of the present invention. According to one embodiment, as set forth in FIG. 1, the device comprises a tubular body 1 of variable length, extending between two end sections, e.g., front end section 2 and rear end section 3, respectively, the direction of forward locomotion of the device in a body cavity of a patient being indicated generally by arrow F. In this manner, the device is movable in a forward and/or reverse direction within the body cavity.

The front end 2 and rear end 3 comprise anchoring means 4, in particular of the clamp type, whereby the device attaches temporarily and alternately to the wall of the body cavity to allow, in a known manner, its locomotion. More particularly the movement of locomotion is achieved as a result of an alternation of elongations and contractions of the tubular body 1, achieved pneumatically, at which the rear end 3 or, respectively, the front end 2, are temporarily anchored to the body cavity via the respective anchoring means 4.

The elongations and contractions of the tubular body 1 are achieved by pressurising or, respectively, depressurising an internal chamber thereof. For this purpose the tubular body 1 may have a bellows configuration, as described for example in patent application WO/02/068035, or, preferably, may be made in an elastic material incorporating a reinforcement structure distributed over its length, substantially rigid in its radial direction and yielding in the axial direction, as described in European patent application no. EP 05425854 in the name of the same Applicant. More particularly the reinforcement structure consists of a spring 19, preferably a pair of consecutive coaxial springs with opposite direction of rotation of the relative coils, incorporated in a silicone tube 20 as described in the aforementioned European patent application.

The anchoring means 4 of the clamp type provided at the front end 2 and rear end 3 of the endoscopic device according to the invention are operated by respective pneumatic actuators 5 that can be made in the same way as the tubular body 1. The rear end 3 is connected to an external control unit by means of a tube 6 conveying the service tubes, including those for admitting compressed air to the tubular body 1 or for creating a vacuum therein, achieving in this way the elongation and the contraction of the tubular body 1 required for locomotion of the device.

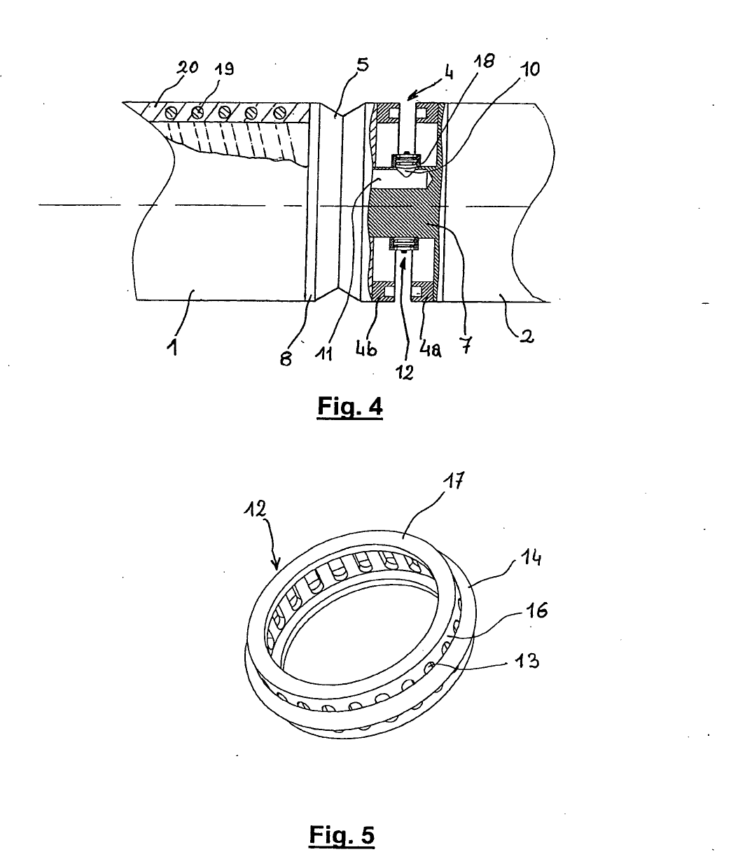

As illustrated in greater detail in FIGS. 2 and 3, relating to the front end 2 (the rear end 3 is equivalent), the clamp anchoring means 4 are formed by a pair of substantially circular jaws 4a and 4b, the first of which is fixed, while the second is mobile in relation to the first. In particular the mobile jaw 4b is mounted slidingly on a multichannel support 7, which can be seen in FIGS. 3 and 4, extending perpendicularly from a connection flange 8 axially connecting the end 2 to the tubular body 1. The multichannel support 7 is illustrated as solid in the drawings for reasons of simplicity, but actually it has several axial channels for the movements actuator fluid, washing of the TV camera, passage of electrical cables and for other accessory services.

The pneumatic actuator 5 of the clamp means 4, schematised as bellows 9 yet which can also be made in silicone material incorporating a helical spring as described in the aforementioned European patent application EP 05425854, is placed between the flange 8 and the jaw 4b. The pressurisation or the depressurisation of the actuator 5 corresponds to an elongation or a contraction of the bellows 9 or, alternatively, of the reinforced elastic pipe, which in turn corresponds to a sliding of the mobile jaw 4b in one direction or in the opposite one and the consequent closure or, respectively, opening of the anchoring means 4.

In the multichannel support 7, in a substantially intermediate position between the jaws 4a and 4b, a suction hole 10 is formed, communicating via a conduit 111 with an external suction unit not shown. At the suction hole 10 on the multichannel support 7 a tubular cylindrical body 12, or filter, is placed, having a plurality of longitudinal apertures 13, evenly distributed circumferentially on its lateral surface. At an intermediate position of the lateral surface of the filter 12 a perimetrical ribbing 14 is formed in a relatively soft material, for example Shore A50 silicone.

As shown in detail in FIGS. 4 and 5, the tubular cylindrical body 12, or filter, is formed by an annular diaphragm 16, on which the longitudinal apertures 13 are formed, and by two flanges 17 extending radially towards the interior of the diaphragm 16 from its perimetrical edges. The filter 12 is force fit mounted on the multichannel support 7, causing one of its radial flanges 17 to abut against a shoulder 18 to align it with the suction hole 10. The two radial flanges 17 also act as spacers between the diaphragm 16 and the surface of the tubular member 7 forming a chamber 15 between them.

Each longitudinal aperture 13 has a width smaller than half the diameter of the suction hole 10 of the conduit 11, so that any debris padding through the filter 12 does not block the suction conduit 11, while debris larger in size than the single aperture may obstruct the same aperture, although it is not detrimental to the suction action.

Thanks to the chamber 15 between the diaphragm 16 of the filter 12 and the multichannel support 7, the suction exerted through the suction hole 10 is distributed substantially evenly in the chamber 15 and, through the apertures 13 of the filter 12, outside of the filter. In this way, when the tissue of the body cavity, drawn between the two jaws 4a, 4b by suction, reaches one of the apertures 13, the suction action is not interrupted until all the apertures have been block by tissue. This situation is the desirable one in that, in this way, the surrounding tissue collapses evenly between the two jaws, achieving an even grip necessary for an effective locomotion step.

The presence of the intermediate perimetrical ribbing 14 on the filter 12 prevents of the tissue of the body cavity from collapsing in the apertures 13, even continuing the suction action, thereby eliminating or, in any case, minimising the risk of damage to the mucosa.

Various modifications and alterations may be appreciated based on a review of this disclosure. These changes and additions are intended to be within the scope and spirit of the invention as defined by the following claims.

What is claimed is:

1. A self-propelled endoscopic device which comprises a tubular body of variable length extending between front end and rear end sections having respective anchors for temporarily and alternately securing the ends to a wall of a patient's body cavity in synch with corresponding axial elongations and contractions of the tubular body that result from injection of compressed air therein and a vacuum created thereby, and pneumatic actuators for activating the anchors, the anchors including a fixed jaw and a mobile jaw joined to the actuators and sliding axially on a multichannel support wherein at least one suction hole is formed, so that a vacuum is generated between the jaws, suitable for drawing the surrounding tissue of the body cavity between them and, in this manner, can be temporarily gripped between the jaws of one end and the other end alternately, wherein coaxially to the support, a relatively evenly perforated diaphragm is positioned at the at least one suction hole, and in spaced relationship from the surface of the support, the width of the perforations being generally smaller than that of the suction hole.

2. The endoscopic device set forth in claim 1, wherein the diaphragm is formed by a relatively hollow cylindrical member having a lateral surface on which a plurality of longitudinal apertures are formed, the width of the apertures being generally smaller than that of the hole and relatively evenly distributed along the surface.

3. The endoscopic device set forth in claim 1, wherein on the cylindrical member at least one perimetrical rib is provided, extending in an intermediate position from the lateral surface.

4. The endoscopic device set forth in claim 3, wherein the perimetrical rib is in a relatively soft material.

5. The endoscopic device set forth in claim 2, wherein the cylindrical member is formed by an annular-like diaphragm and a plurality of flanges extending generally radially and inwardly from the perimetrical edges of the diaphragm.

6. The endoscopic device set forth in claim 1, wherein the cylindrical member is force fit mounted to the multichannel support and abuts a shoulder on the support.

7. The endoscopic device set forth in claim 1, wherein the longitudinal apertures have a width smaller generally than about half the diameter of the hole.

8. The endoscopic device set forth in claim 2, wherein the tubular body with variable length comprises an elastic material incorporating a reinforcing structure distributed over its length and substantially rigid in a radial direction while yielding in an axial direction.
